(19) United States
(12) Patent Application Publication
(10) Pub. No.: US 2013/0196876 A1
(43) Pub. Date: Aug. 1, 2013

(54) DIFFERENTIAL DIAGNOSIS OF PANCREATIC ADENOMAS

(71) Applicants: Jens Habermann, Celle (DE); Timo Gemoll, Grossenbrode (DE); Uwe Johannes Roblick, Lubeck (DE)

(72) Inventors: Jens Habermann, Celle (DE); Timo Gemoll, Grossenbrode (DE); Uwe Johannes Roblick, Lubeck (DE)

(73) Assignee: UNIVERSITATSKLINIKUM SCHLESWIG-HOLSTEIN, Kiel (DE)

(21) Appl. No.: 13/876,281

(22) PCT Filed: Sep. 28, 2011
(86) PCT No.: PCT/EP11/04847

§ 371 Date: Mar. 27, 2013

(30) Foreign Application Priority Data
Oct. 1, 2010 (DE) 10 2010 047 069.4

(51) Int. Cl. G01N 33/68

(52) U.S. Cl. CPC G01N 33/6893
USPC 506/9

(57) ABSTRACT

The invention relates to a method for the diagnosis of pancreatic adenomas, to biomarkers for use in said method, to kits for carrying out the method, and to the use of said method for detecting pancreatic adenomas. The following markets are used: KCNABT, CIORF77, SPSB2, MED9, STK40, ITGB3BP, SCEL, PDPK1, DUSP15, MED19, NAP1L3, TMOD3, CSPP1, TMOD2, INPP5A and IGHG.

The invention relates to a method for diagnosis of pancreatic adenomas, biomarkers for use in this method, kits for performing the method and the use of this method for detection of pancreatic adenomas and for assessing the progression to pancreatic cancer.

In the Western industrialized countries, the pancreatic ductal adenocarcinoma is a tumor disease with an increasing incidence and is with a 5-year survival rate of 1% in 4. to 5. place among the tumor related causes of death (Cludjonsson, B. (1987) Cancer of the pancreas. 50 years of surgery. Cancer 60, 2284-2303; Parker, S. L., Tong, T., Bolden, S., and Wingo, P. A. (1997) Cancer statistics, 1997. CA Cancer J Clin 47, 5-27). Men are affected more often than women and ⅔ of all patients at diagnosis have already passed the age of 60 (Urrutia, R. (1997) Exploring the role of homeobox and zinc finger proteins in pancreatic cell proliferation, differentiation, and apoptosis. Int J Pancreatol 22, 1-14). The combination of early-onset metastasis and often only insufficient diagnostic and treatment options are the main reasons for the particularly unfavorable prognosis. Because at the time of diagnosis the lymph notes and peripheral nerves are affected by the tumor cells in up to 50% of patients (Takahashi, T, ishikura, I L, Motohara, T., Okushiba, S., Dohke, M., and Katoh, H. (1997) Perineural invasion by ductal adenocarcinoma of the pancreas. J Surg Oncol 65, 164-170), only 10-20% of all patients can be treated by radical resection of the primary tumor (Zamboni, G., Capelli, P., Pesci, A., Beghelli, S., Luttges, J., and Kloppel, G (2000) Pancreatic head mass: what can be done? Classification: the pathological point of view. JOP 1, 77-84). Routine clinical diagnosis includes physical examination, sonography and computer tomography. If computer tomogram and ultrasound do not provide adequate information, the magnetic resonance imaging or, more rarely, the positron-emission-tomography can be employed as further imaging methods. A definite diagnosis confirmation is nowadays, however, only possible by examining of tissues under the microscope. This tissue is attained by using the endoscopie, endosonography, ultrasound examination or the computer tomography by puncture through the skin, mostly under local anaesthetic. All methods for diagnosis of pancreatic carcinoma are usually done only when patients already show symptoms that indicate an advanced stage of disease.

Thus, it is not possible up to now, due to the routine diagnosis to make preoperatively a distinction between benign pancreatic changes, such as pancreatic adenomas, and pancreatic carcinoma. A distinction would allow to plan the surgical procedure more detailed accordingly: the pancreatic adenoma would be preventive surgically removed. This approach would not, however, be required to be subject to oncology guidelines, which means that, in the best case, parts of the pancreas could even be preserved, the surgical procedure would he less aggressive, and the patients would thus have a shorter covalence period.

There is a wealth of evidence for the existence of an immune response against cancer in humans, which could be demonstrated by the presence of autoantibodies in sera of cancer patients. Autoantigenes change thereby before or during tumor formation and can subsequently cause an immune response via autoantibodies (Chatterjee, M., et al. (2006) Diagnostic markers of ovarian cancer by high-throughput antigen cloning and detection on arrays. Cancer Res 66, 1181-1190; Hudson, M. E., Pozdnyakova, I., Haines, K., Mor, G., and Snyder, M. (2007) identification of differentially expressed proteins in ovarian cancer using high-density protein microarrays. Proc Natl Acad Sci U S A 104, 17494-17499; Soussi, T. (2000) p53 Antibodies in the sera of patients with various types of cancer: a review. Cancer Res 60, 1777-1788).

Studies on autoantibodies in the pancreatic carcinoma were also completed. Thus, two-dimensional gel electrophoresis, Western blots and mass spectrometry for identification of antibodies against pancreatic cancer and pancreatitis have been used (Hong, S. H., Misek, D. E., Wang, H., Puravs, E., Giordano, T. J., Greenson, J. K., Brenner, D. E., Simeone, D. M., Logsdon, C. D., and Hanash, S. M. (2004) An autoantibody-mediated immune response to calreticulin isoforms in pancreatic cancer. Cancer Res 64, 5504-5510). Calreticulin isoforms have been here described as possible markers for early diagnosis. This approach allows, in contrast to the protein array, the analysis of proteins in their natural state and environment. However, one is limited to the resolution of the two-dimensional gel electrophoresis of about 1500 proteins.

Antibody microarrays have been also already successfully used in the pancreatic cancer research. Such microarrays allow the detection of specific serum autoantibodies to a very large number of targets simultaneously. The arrays can be used with a high throughput in order to determine patterns of antigenes recognized by autoantibodies during the course of disease. Gnjatic et al. have used the v4.0 version of the Protoarray of Invitrogen (about 8000 targets) for comparison of pancreatic cancer patients with a control group (n=113). 29 serum proteins that could serve as biomarkers for classification of certain pancreatic tumor stages were identified (Gnjatic, S., Ritter, E., Buehler, M. W., Giese, N. A., Brors, B., Frei, C., Murray, A., Halama, N., Zornig, I., Chen, Y. T., Andrews, C., Ritter, O., Old, L. J., Odunsi, K., and Jager, D. (2010) Seromic profiling of ovarian and pancreatic cancer, Proc Natl Acad Sci U S A 107, 5088-5093).

Detailed studies on human tissue samples as well as new mouse models have revealed the early pancreatic tumor development from the precursor lesions through to the invasive carcinoma (Kleeff, J., Michalski, C. W. and Friess H. (2008) Molekulare Aspekte des Pankreaskarzinoms—ein Update. Onkopipeline 1, 57-62). However, no tumor-specific autoantibodies were identified up to now, which are characteristic for early stages of pancreatic tumors, particularly pancreatic adenomas, and may thus serve as potential biomarkers for differential diagnosis. Thus, at present there are no in vitro screening tests which allow a distinction between pancreatic adenomas and pancreatic carcinomas. Accordingly, there is a need for innovative screening and therapy methods which may lead to an earlier diagnosis and a distinctly improvement in prognosis of pancreatic cancer patients.

The technical problem of the present invention is thus to provide a method for diagnosis of pancreatic adenoma and for distinction between pancreatic adenoma and pancreatic carcinoma.

The technical problem is solved by a method for in vitro diagnosis of a pancreatic adenoma with the steps:

(a) incubating a sample containing antibodies taken from an individual with at least one protein or protein fragment encoded by one of the genes selected from a first group of genes consisting of KCNAB1, C1ORF77, SPSB2, MED9, STK4O, ITGB3BP, SCEL, PDPK1, DUSP15 and MED19 and/or at least one protein or protein fragments encoded by one of the genes selected from a second group of genes consisting of NAP1L3, TMOD3, CSPP1, TMOD2, INPP5A and IGHG1,

(b) determining the amount of antibodies bound to the at least one protein or the protein fragment; and

(c) comparing the amount of antibody from the sample bound to the at least one protein or protein fragment with the average amount of antibody present in healthy individuals against the at least one protein or protein fragment, wherein:
a lower amount of the specific antibody directed against the at least ne protein or protein fragment encoded by the first group of genes, and/or
a higher amount of the specific antibody directed against the at least one protein or protein fragment encoded by the second group of genes indicates the presence of a pancreatic adenoma,

In an embodiment, the at least one protein or protein fragment is encoded by TMOD3 or INPP5A from the second group of genes.

In an embodiment, the sample is blood or serum.

In an embodiment, the at least one protein or protein fragment is bound to a carrier,

In an embodiment, the determining of the amount of antibodies bound to the at least one protein or protein fragment is carried out using labelled anti-human immunoglobulin antibodies.

In an embodiment, the anti-human immunoglobulin antibodies are labelled with an enzyme, biotin, a radioactive isotope or a fluorescent dye.

The technical problem of the present invention is further solved by a kit for performing of the above-described method,

characterized in that

at least one protein or protein fragment encoded by one of the genes selected from a first group of genes consisting of KCNAB1, C1ORF77, SPSB2, MED9, STK40, ITGB3BP, SCEL, PDPK1, DUSP15 and MED19 and/or at least one protein or protein fragment encoded by one of the gene selected from a second group of genes consisting of NAP1L3, TMOD3, CSPP1, TMOD2,,INPP5A and IGHG1; and

at least one labelled anti-human immunoglobulin antibody.

In an embodiment, the at least one protein or protein fragment is encoded by TMOD3 or INPP5A.

In an embodiment; the protein or protein fragment is immobilized on a carrier.

In an embodiment, the anti-human immunoglobulin antibody is labelled with an enzyme, biotin, a radioactive isotope or a fluorescent dye.

The technical problem of the present invention is further solved by the use of at least one protein or protein fragment, which is encoded by a gene selected from the group consisting of KCNAB1, C1ORF77, SPSB2, MED9, STK40, ITGB3BP, SCEL, PDPK1, DUSP15, MED19, NAP1L3, TMOD3, CSPP1, TMOD2, INPP5A and IGHG1, or at least one autoantibody directed against that gene as diagnostic marker for the diagnosis of a pancreatic adenoma.

In an embodiment, the at least one protein or protein fragment is encoded by TMOD3 or INPP5A.

According to the invention, the technical problem is solved by a method for in vitro diagnosis of a pancreatic adenoma, wherein the amount of an autoantibody against at least one of the proteins encoded by the genes shown in Table 1 and/or Table 2 is determined in a sample of biological origin, particularly in the serum of an individual, The amount of an autoantibody in this sample is compared with the amount of the corresponding autoantibody, which is normally present in healthy patients. in case this is a gene shown in the Table 1, an increased amount of the autoantibody is an indication of the presence of a pancreatic adenoma. In case this is one of the genes shown in the Table 2, a decreased amount of the autoantibody is an indication of the presence of a pancreatic adenoma.

The invention is based on a study for the identification of autoantibodies and tumor-associated antigens in pancreatic adenomas as precursors of carcinomas, whereby it has been determined which autoantibodies in the pancreatic adenoma have an altered level of occurrence comparing to healthy patients. For this purpose, a comparative study of serum samples from patients with pancreatic adenomas (n=5), pancreatic carcinomas (n=10) and healthy patients (n=10) has been carried out (Example 1) using high-density protein microarrays (Invitrogen, v5.0;>9000 human proteins). When comparing the obtained profiles a total of 17 proteins have been detected statistically significant between pancreatic adenoma and healthy controls (p<0.05; Tables 1 and 2). Thereby, 7 antibodies have shown an upregulation (Table 1) and 10 a downregulation (Table 2) in pancreatic adenomas. Autoantibodies which have shown a significant difference in the comparison between pancreatic carcinomas and healthy controls (n=9) as well as in the comparison between pancreatic adenomas and carcinomas (n=9) were excluded from the results.

In this way, pancreatic-associated autoantibody repertoire and corresponding tumor-associated antigenes have been identified. The altered presence of these proteins is causally linked to the pancreatic tumor. Compared to previous studies, a variety of new proteins has been detected using the new version of the protoarray of the Invitrogen, which may serve as potential biomarkers for an early detection of the pancreatic carcinoma. This was confirmed by a specific cohort of patients, reflecting the carcinogenesis of healthy individuals from pancreatic adenomas up to pancreatic carcinomas.

Significant expression differences between pancreatic adenomas and healthy controls could be also confirmed for two proteins (TMOD3 and INPP5A) using an independent statistical method. The quantified signals of the ProtoArrays from background were here subtracted, normalized and log2 transformed. The Mann Whitney U Test has served for detection of differently regulated targets.

TMOD3 and INPP5A have also been determined by two different statistical methods and therefore appear to be particularly well suited for distinguishing pancreatic adenomas from healthy controls.

Surprisingly, it was not known from any of the 17 proteins found that they associate with pancreatic tumor, Thus, the presence of specific antibodies for pancreatic tumors and particularly for pancreatic adenomas is confirmed, The new individual pattern of proteins according to the present invention allows to detect and to diagnose pancreatic carcinomas with higher specifity and sensivity than the previous known serum biomarkers in early stages of carcinoma development. A large number of adenoma and carcinoma carriers could thereby be detected early enough so that a treatment with lower morbidity and lower costs is possible.

The method according to the present invention is performed in vitro. The sample of biological material, which is taken from a patient for diagnosis of the pancreatic adenoma is preferably a blood sample or a serum sample containing proteins. Each biomarker protein used comprises preferably a full-length molecule or a fragment thereof, which is suitable for detection of anti-biomarker-antigene autoantibodies. Preferably, the biomarker according to the present invention are bound to a carrier. Such a solid carrier can be, for example, an ELISA plate, a magnetic bead or a blot film, e. g., a nitrocellulose sheet. The biomarker proteins can be attached directly to the carrier or coupled to a carrier, e. g., via antibodies in particular antibodies against a tag associated with biomarker proteins, such as glutathione-S-transferase. These antibodies are not human antibodies in order to prevent cross-reactions with secondary antibodies. In a preferred embodiment of the present invention, the binding of the autoantibodies of the invention to their antigenes is examined by contacting the carrier with labelled anti-immunoglobulin antibodies against antibodies of the patient, and detecting the labelled antibodies. The anti-immunoglobulin antibodies labelled with an enzyme, such as alkaline phosphatase or horseradish peroxidase, biotin, a radioactive isotope or a fluorescent dye, such as fluoresceinisothiocyanat (FITC) or phycoerythrin (PE) are preferred. The examination may he conducted in a blot, e. g., a dot blot or a Western blot, an ELBA (enzyme linked immunosorbent assay), a RIA (radioimmunoassay), a FACS (fluorescence activated cell sorting) examination or also in a liquid phase detection system.

A kit for performing of the method according to the present invention is also provided. Preferably, this kit comprises biomarker proteins and labelled anti-immunoglobulin antibodies of the present invention. Each him-parker protein used preferably comprises a full-length molecule or a fragment thereof, which is suitable for detection of anti-biomarker-antigene autoantibodies. The labelled antibodies contained in the kit may be labelled with an enzyme; such as alkaline phosphatase or horseradish peroxidase, biotin, a radioactive isotope or a fluorescent dye, such as FITC or PE.

Finally, the invention also relates to the use of autoantibodies against the biomarker proteins of the present invention as diagnostic markers in patients with pancreatic adenoma and thus the use of a kit described herein for determination of autoantibodies against the biomarker proteins of the present invention as diagnostic markers for the survival prognosis in patients with pancreatic adenoma.

The invention is described in more detail by the following example.

### EXAMPLE 1

All sera were processed by an identical procedure. Blood samples were stored at room temperature for maximum 30 min to allow clotting, and then centrifuged at 3000× g for 10 min at 4° C. Until further processing, the sera were stored at −180 CC. 25 serum samples (10 sera of patients with pancreatic tumors, 5 patients With a pancreatic adenoma and 10 healthy subjects) were tested and analyzed by the Human ProtoArray™ v5,0 (Invitrogen), according to the manufacturer's recommendations: All ProtoArrays were equilibrated 20 ruin at 4° C. and then incubated with gentle shacking with blocking buffer (1% BSA, 0.5 mM DTT, 5% glycerol, 0.05% Triton X-100 in PBS) for 1 h at 4° C. Subsequently, the arrays were covered with 10 μL serum (1:500 dilution) for 90 min at 4° C. and washed (1% BSA in 0.1% Tween 20, PBS). Human bound antibodies were detected after incubation for 90 min at 4° C. with Alexa Fluor (647-goat anti-human IgG (Invitrogen), 12000 dilution). To quantify the signals, an Axon 4200AL scanner (Molecular Devices) with GenePix Pro 5.1 image analysis software Molecular Devices) was used.

TABLE 1
Autoantibodies downregulation in pancreatic adenomas compared to healthy
controls

| Gene | NCBI ID | Description | P-value |
|---|---|---|---|
| KCNAB1 | NM_172159.2 | Potassium voltage-gated channel, shaker-related | 0.00366 |
| | | subfamily, beta member 1 (KCNAB1), transcript variant 3 | |
| C1ORF77 | BC002733.2 | Chromosome 1 open reading frame 77 (C1orf77) | 0.01698 |
| SPSB2 | NM_032641.1 | SpIA/ryanodine receptor domain and SOCS box containing | 0.01865 |
| | | 2 (SPSB2) | |
| MED9 | NM_018019.1 | Mediator complex subunit 9 (MED9) | 0.01865 |
| STK40 | NM_032017.1 | Serine/threonine kinase 40 (STK40) | 0.02198 |
| ITGB3BP | NM_014288.2 | Centromere protein R | 0.04196 |
| SCEL | BC047536.1 | Sciellin (SCEL) | 0.04196 |
| PDPK1 | NM_002613.3 | 3-phosphoinositide dependent protein kinase-1 (PDPK1), | 0.04196 |
| | | transcript variant 1 | |
| DUSP15 | BC056911.1 | Dual specificity phosphatase 15 (DUSP15) | 0.04196 |
| MED19 | NM_153450.1 | Mediator complex subunit 19 (MED19) | 0.04196 |

TABLE 2
Autoantibodies upregulation in pancreatic adenomas compared to healthy
controls

| Gene | NCBI ID | Description | P-value |
|---|---|---|---|
| NAP1L3 | BC34954.2 | Nucleosome assembly protein 1-like 3 (NAP1L3) | 0.01698 |
| TMOD3 | NM_014547.3 | Tropomodulin-3 | 0.01698 |
| CSPP1 | NM_024790.2 | Centrosome and spindle pole associated protein 1 | 0.01698 |
| | | (CSPP1), transcript variant 2 | |
| TMOD2 | BC036184.1 | Tropomodulin-2 | 0.01865 |
| TMOD2 | NM_014548.2 | Tropomodulin 2 (neuronal) (TMOD2) | 0.01865 |
| INPP5A | BC096280.2 | Type I inositol-1,4,5-trisphosphate 5-phosphatase | 0.02198 |
| IGHG1 | BC014667.1 | Immunoglobulin in heavy constant gamma 1 (G1m | 0.02198 |
| | | marker) (IGHG1) | |

1. A method for in vitro diagnosis of a pancreatic adenoma comprising the steps:
(a) incubating a sample containing antibodies taken from an individual with at least one protein or protein fragment encoded by one of the genes selected from a first group of genes consisting of KCNAB1, C1ORF77, SPSB2, MED9, STK40, ITGB3BP, SCEL, PDPK1, DUSP15 and MED19 and/or at least one protein or protein fragment encoded by one of the genes selected from a second group of genes consisting of NAP1L3, TMOD3, CSPP1, TMOD2, INPP5A and IGHG1,
(b) determining the amount of antibody bound to the at least one protein or the protein fragment; and
(c) comparing the amount of antibody from the sample bound to the at least one protein or protein fragment with the amount of antibody present in healthy individuals on average against the at least one protein or protein fragment, wherein:
a lower amount of the specific antibody directed against the at least one protein or protein fragment encoded by the first group of genes, and/or
a higher amount of the specific antibody directed against the at least one protein or protein fragment encoded by the second group of genes indicates the presence of a pancreatic adenoma.

2. The method according to claim 1, wherein the at least one protein or protein fragment is encoded by TMOD3 or INPP5A.

3. The method according to claim 1, wherein the sample is blood or serum.

4. The method according to claim 1, wherein the at least one protein or protein fragment is bound to a carrier.

5. The method according to claim 1, wherein the determining of the amount of antibody bound to the at least one protein or protein fragment is carried out using labelled anti-human immunoglobulin antibodies.

6. The method according to claim 5, wherein the anti-human immunoglobulin antibodies are labelled with an enzyme, biotin, a radioactive isotope or a fluorescent dye.

7. A kit for performing of the method according to claim 1, comprising
at least one protein or protein fragment encoded by one of the genes selected from a first group of genes consisting of KCNAB1, C1ORF77, SPSB2, MED9, STK40, ITGB3BP, SCEL, PDPK1, DUSP15 and MED19 and/or at least one protein or protein fragment encoded by one of the genes selected from a second group of genes consisting of NAP1L3. TMOD3, CSPP1, TMOD2, INPP5A and IGHG1; and
at least one labelled anti-human immunoglobulin antibody.

8. The kit according to claim 7, wherein the at least one protein or protein fragment is encoded by TMOD3 or INPP5A.

9. The kit according to claim 7, wherein the protein or protein fragment is immobilized on a carrier.

10. The kit according to claim 7, wherein the anti-human immunoglobulin antibody is labelled with an enzyme, biotin, a radioactive isotope, or fluorescent dye.

11. A use of at least a protein or protein fragment, which is encoded by a gene selected from the group consisting of KCNAB1, C1ORF77, SPSB2, MED9, STK40, ITGB3BP, SCEL, PDPK1, DUSP15, MED19, NAP1L3, TMOD3, CSPP1, TMOD2, INPP5A and IGHG1, or at least one autoantibody directed against that gene as diagnostic marker for the diagnosis of a pancreatic adenoma.

12. The use according to claim 11, wherein the at least one protein or protein fragment is encoded by TMOD3 or INPP5A.
